(19) United States
(12) Patent Application Publication
(10) Pub. No.: US 2025/0243452 A1
(43) Pub. Date: Jul. 31, 2025

(54) METHOD AND SYSTEM FOR MERGING BIO-ELECTROSPRAYING, CELL ELECTROSPINNING MERGED WITH 3D MULTIMATERIAL MICROFLUIDIC BIOPRINTING FABRICATE HUMAN TISSUES FOR DRUG DISCOVERY APPLICATIONS

(71) Applicant: Ourobionics BV, Arnhem (NL)

(72) Inventors: Suwan N. Jayasinghe, Watford (GB); Stephen G. Gray, Utrecht (NL); Ali Leylavi Shoushtari, Ede (NL)

(73) Assignee: Ourobionics BV, Arnhem (NL)

(21) Appl. No.: 18/424,913

(22) Filed: Jan. 29, 2024

(51) Int. Cl. C12N 5/00; C12M 1/00; C12M 1/36; C12M 3/00; C12M 3/06

(52) U.S. Cl. CPC C12N 5/0062; C12M 21/08; C12M 23/16; C12M 29/06; C12M 41/48

(57) ABSTRACT

The present invention provides a method and system for fabricating complex human tissues by integrating bio-electrospraying, cell electrospinning merged with 3D multi material microfluidic bioprinting, and future additions of magnetic and acoustic levitation technologies. The fabricated tissues serve as an alternative to animal testing in drug discovery processes, aiding in the prediction of human physiological responses to various drug compounds.

### BACKGROUND OF THE INVENTION

### Field of the Invention

The present invention relates to systems and methods for fabricating complex human tissues by integrating bio-electrospraying, cell electrospinning merged with 3D multi material microfluidic bioprinting. The implementation of this system enriches and enlarges our potential in tissue fabrication, allowing the construction of multi-materials and materials graded structures from the molecular level upwards. The fabrication techniques are enhanced by the future additions of magnetic and acoustic levitation technologies. In an embodiment, the fabricated tissues can serve as an alternative to animal testing in drug discovery processes. Accordingly, the systems and methods can aid in the prediction of human physiological responses to various drug compounds.

With the evolution of regenerative medicine, the demand for precision-engineered human tissues has grown exponentially. The current models, though beneficial, often fail to mimic the intricacies of human physiology, leading to inaccurate drug interaction results and an over-reliance on animal testing.

### The State of the Art of Tissue Bioprinting Technologies

3D bioprinting is one of the techniques enabling the creation of 3D cell cultures that have been very promising with the establishment of 3D printers capable of depositing hydrogels with cells into a 3D structure. 3D cell culture technology is an in vitro technique in which cells grow in an artificially created environment, which resembles the in vivo environment. This technique stimulates cells to differentiate, proliferate, and migrate by interacting with their three-dimensional surroundings. With the development of 3D bioprinters, there has been a huge increase in research related to 3D cell culturing. Unfortunately, the outcome of this research has mostly been limited to publications rather than applications.

The reason that the bioprinting sector has not been able to live up to its expectations, is due to technological limitations. The current technology has reached its ceiling with regard to creating alternative solutions for the safety and efficacy testing of new treatment methods. Unfortunately, with what is currently available, the industry is not able to create solutions that are able to provide a better model than the current animal models. This has left the technology merely being used for research purposes only. The issues with the technologies are related to low cell viability (40-70%), low functionality, low throughput, limited complexity and slow speed (1 cm³ of tissue in 30 minutes).

Techniques such as the widely used extrusion bioprinting create stress during the process. This causes both cell death and cell deformation and results in loss of functionality. The protection of the molecular integrity of the cell with this technology remains a big question mark. Even in the scenario that the available technologies would have the ability to create viable and functional applications, scalability would be severely challenged due to the speed. There are some novel technologies now available (such as Laser-assisted bioprinting, stereolithography, volumetric and acoustic bioprinting). Although they do tackle one or more of the issues, they (especially on their own) fail to tackle all of them leaving them to become just another research purpose tool only. Moreover, they introduce new limitations such as the specific use of certain biomaterials that tend to be harmful for cells. None of the technologies available offers the ability to create any level of complexity in the 3D structure allowing the use of different compositions of biomaterials (Cells, hydrogels/bioinks, growth factors, genes, etc.) within the same structure. It is with these limitations in mind that the present invention was developed.

### SUMMARY OF THE INVENTION

The present invention relates to systems and methods for fabricating complex human tissues by integrating bio-electrospraying, cell electrospinning merged with 3D multi material microfluidic bioprinting. The fabrication techniques are enhanced by the future additions of magnetic and acoustic levitation technologies. In an embodiment, the fabricated tissues can serve as an alternative to animal testing in drug discovery processes. Accordingly, the systems and methods can aid in the prediction of human physiological responses to various drug compounds.

### BRIEF DESCRIPTION OF THE SEVERAL VIEWS OF THE DRAWINGS

FIG. 1 depicts a schematic presentation of the microfluidic-based bio-printer of the invention.

FIG. 2 depicts the principles and components of the microfluidic circuit as well as the flow rate/viscosity controller.

### DETAILED DESCRIPTION OF THE INVENTION

The present invention relates to systems and methods for fabricating complex human tissues by integrating bio-electrospraying, cell electrospinning merged with 3D multi material microfluidic bioprinting. The fabrication techniques are enhanced by the future additions of magnetic and acoustic levitation technologies. In an embodiment, the fabricated tissues can serve as an alternative to animal testing in drug discovery processes. Accordingly, the systems and methods can aid in the prediction of human physiological responses to various drug compounds.

In an embodiment, the present invention relates to being able to grow/fabricate complex tissue that can be used for a plurality of applications such as drug testing. In an embodiment, the present invention relates to being able to optimize fabrication conditions so as to generate superior results relative to the tissues of the prior art.

Desired state: what technological goals are supposed/desired to achieve? To have a functional tissue the following parameters should be met:

### Cell Viability and Functionality:

It is important to fabricate cells that are viable and this is an area in which improvements in fabrication outcomes can be attained. Ensuring that the cells used in the process remain viable and maintain their functionality throughout the fabrication process is important. The improvements in bio-electrospraying and cell spinning should focus on minimizing cell damage and optimizing cell survival.

### Incorporating Multiple Cell Types:

Human tissues are composed of multiple cell types that require different microenvironments to grow and proliferate. Multimaterial approaches should enable the precise placement of these different cell types and biomaterials within the 3d matrix. Thus, magnetic and acoustic levitation apparatuses can be used to optimize this parameter.

### Vascularization:

For human tissue to be fully functional, it requires a vascular network to supply nutrients and remove waste products. For example developing techniques for the creation of blood vessels within the skin construct is a significant challenge. Providing precise three-dimensional printing allows for the cells to be ideally vascularized.

### Tissue Complexity

The capability to construct complex tissue structure is essential for sensing and responding to external stimuli. For instance, incorporating neurons and ensuring their proper connection with other components is a key challenge. Providing precise three-dimensional printing allows for the neuron cells to be ideally positioned as needed in these complex tissues.

### Biomechanical Properties:

human tissues should have appropriate mechanical properties to withstand the stresses and strains they encounter in everyday life. The mechanical properties of the human tissue construct need to be carefully engineered. Providing the ideal mix of the different types of cells in the correct configuration will provide mechanical strength to the complex tissues.

### Biomaterial Selection:

The choice of biomaterials for the scaffolds and matrices used in bio-electrospraying and cell spinning is important. Researchers should focus on materials that closely mimic the extracellular matrix of native tissue. The use of these materials also helps with mechanical strength and cell/tissue viability.

### Scale-Up and Clinical Translation:

Techniques need to be scalable for clinical use. Transitioning from small lab-scale constructs to larger, clinically relevant sizes while maintaining quality is a significant challenge. The use of the correct materials, their correct incorporation into tissues, and providing sufficient mechanical strength all aid in the ability to scale up the tissues for clinical use.

### Technology Gap: What is Missing Between Current State and the Desired State:

There currently appears to be a gap between the tissues that are created using the technology of the prior art and satisfying the above-identified criteria. Bio-electrospraying and cell electrospinning improved cell viability and also led to a good improvement in cell functionality (Jayarajan et. al, 2023). To meet an optimized functional tissue systems and methods need to be developed (and are described herein) that allow the incorporation of multiple cell types, create vascularization, tissue appendages e.g. in case of skin hair follicles, sweat glands, and sebaceous glands and allow nerve integration while obtaining the targeted biomechanical properties in the tissue. To meet these conditions, the present invention contemplates the use of electrospun and electrospray multiple biomaterials and cells with adjustable gradients of biomaterial deposition. The currently available bioprinting methods employ multiple independent print-heads to achieve such a purpose which practically turns the bioprinting into a batch process comprising enormous shifts between print-heads. This process not only is a slow process of printing but using this process also precludes one the ability to create and mimic the natural gradient in tissues.

How Merging Bio-Electrospraying and Cell Electrospinning with Microfluidic Technology and Melt Electro Writing Will Close the Technology Gap

The present invention contemplates the use of microfluidic circuits. Microfluidic circuits are miniature systems that manipulate tiny volumes of fluids, used in diverse applications. One important function that can be attained by microfluidic circuits is generating gradient mixtures by precisely controlling the combination of different fluids at varying ratios within the microchannels. It allows the manufacture of multi-materials and materials graded structures at a molecular level. These gradients, which can be linear, exponential, or customized, are instrumental in research areas like drug testing, cell culture, and chemical analysis. This enables the study of how cells, molecules, or materials respond to changing environments with high precision and miniaturization, making microfluidic circuits a powerful tool in scientific investigations.

Integrating a microfluidic circuit into the bio-electrospraying and cell spinning process would introduce a novel capability of gradient bio-electrospraying. It allows the generation of a gradient cell concentration that helps with manufacturing a more accurate disease model (i.e., via generating a desired bacterial gradient in tissue), more accurately generating/mimicking the natural biomechanical properties of tissue. For instance, it has been found that the cell concentration plays a key role in having a growing uniform epidermis after bio-electrospraying human skin cell culture (see, for example, Jayarajan et. al, 2023). Therefore, microfluidic circuits and systems that comprise them are an excellent choice to automatically adjust and control the cell concentrations in hydrogel solvents prior to electrospraying/spinning.

Moreover, melt-electro writing (MEW) allows the pores to be size controlled, which enables membranes to be printed at any given porosity. Having a gradient of biomaterial deposition and membranes with specific porosity allows the adjustment of cell microenvironments, which is one key to steering cell migration after printing.

In an embodiment, the present invention introduces a breakthrough method and system for fabricating human tissues of unparalleled complexity and precision. By synergistically integrating the following technologies one can attain complex human tissue that heretofore was unrealized:
1. 3D Bio-Electrospraying: This technology dispenses cells and biological materials with an electric field, ensuring cell viability and precise deposition.
2. 3D Cell Electrospinning: This novel technique produces nanofiber scaffolds with cellular components, providing an excellent environment for cell growth and differentiation.
3. Melt/cell Electro-Writing: This novel technique allows one to print nano scaffolds out of biomaterials embedded with cells thereby providing an ideal scaffold for cell (and tissue) growth.
4. Merging the above technologies with 3D multi-material microfluidic gradient bioprinting allows for the dynamic control over gradient of the biomaterial deposition. This also results in enabling the changes in the cell microenvironment (which allows steering cell migration). This also allows for advantageous printing of subcutaneous tissue structures with a gradient in mechanical properties (e.g., stiffness and viscosity—soft to rigid and low-to-high viscosity respectively). These tend to be prerequisites for the creation of vascularized and multi-layered structures by dispensing cells in a highly controlled manner, mimicking natural tissue arrangements, and providing the tissues with all of the advantages one sees in naturally created tissue.
5. The present invention also incorporates the use of magnetic and acoustic levitation technologies: These allow for the manipulation and positioning of cells without physical contact, thereby not only preserving cellular integrity and promoting organic tissue formation, but reducing the mechanical stress that may otherwise be placed upon the cells.
6. To the inventors knowledge, this is the first time that the fully integrated and multiplexed systems as disclosed herein can produce tissues that emulate the detailed architectural and functional attributes of native human tissues.

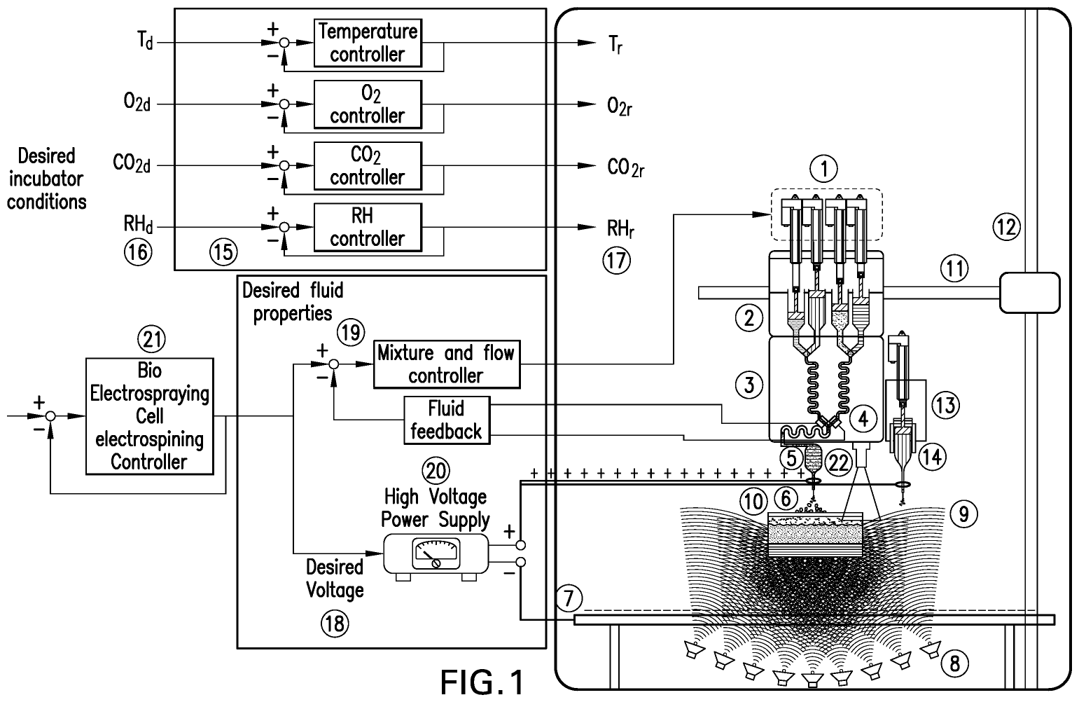

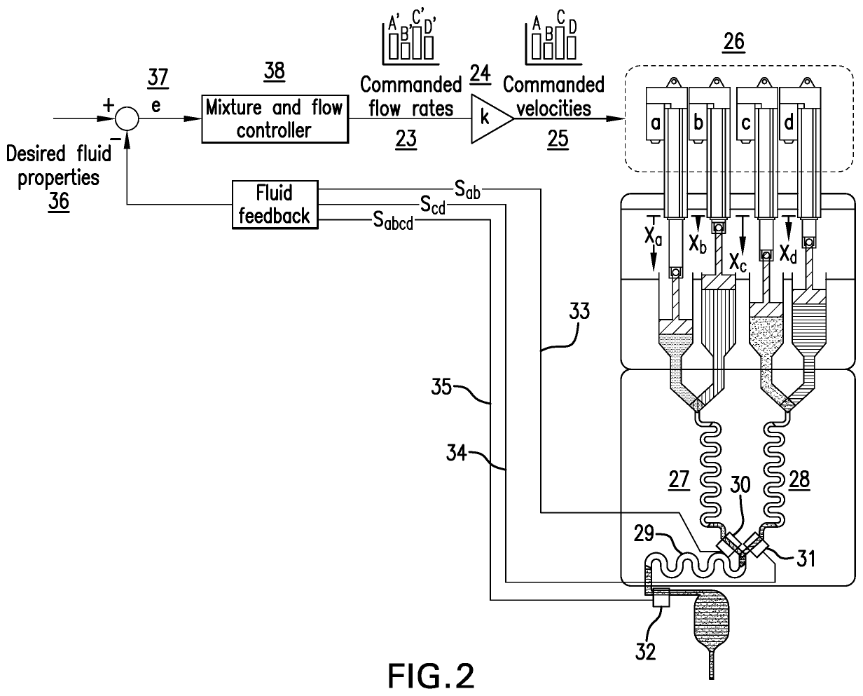

FIG. 1 depicts one embodiment showing a schematic presentation of a microfluidic-based bio-printer that allows one to attain the tissues discussed above. In an embodiment, the device of the present invention as shown in FIG. 1 comprises seven modules. These are:
Microfluidic printhead (MP): comprising a set of linear actuators (1) that through mechanical work independently allows the biomaterials in syringes to flow (2). Then, passing the flow of biomaterials through a microfluidic mixer circuit (3) would blend them to a homogenous suspension with any desired cell concentration (5).
FIG. 2 shows the details of this module. The microfluidic mixer comprises at least one primary and at least one secondary mixing channel. Depending on the number of the input biomaterials, there are different numbers of mixing channels. Having four biomaterials here, the apparatus would be set up to have two primary channels (FIG. 2): with primary channel (27) in charge of blending biomaterials a and b and channel (28) which mixes materials c and d. Subsequently, the secondary channel (29) blends the initial mixes of ab and cd. The fluidic property of the resulting mix abcd is adjusted on-time using a closed loop feedback controller. In particular, the fluid properties e.g. viscosity and flow rate of the initial blends ab and cd and of the secondary blend abcd are measured via fluidic sensors (30), (31) and (32) respectively. These sensors generate three feedback signals S_{ab} (33) S_{cd} (34) and S_{abcd} (35) which are used by the mixture and flow controller to set the final fluid properties on the desired value (36).
The controller (38) presented in FIG. 2, receives the error signal and calculates the commanded flow rate signal (23). The error signal is defined as the difference between current viscosity and the desired one. The generated flow rate command would be converted to a velocity profile signal (25) for the actuators using the proportional coefficient k (24). The logic underlying this conversion is: the output flow rate of mixing channels depends on how fast the biomaterials inside the syringes are pushed. The actuators a, b, c and d are in charge of pushing the syringes with velocities

$${V}_{a} = \frac{d}{dt} {x}_{a} , {V}_{b} = \frac{d}{dt} {x}_{b} , {V}_{c} = \frac{d}{dt} {x}_{c} and {V}_{d} = \frac{d}{dt} {x}_{d}$$

respectively. Which xₐ, x_{b}, x_{c} and x_{d} are displacements of the actuators a-d respectively. An in-built velocity controller (26) is in charge of tracking the velocities of the actuators.
Biomaterial accelerator module (BAM): comprises a high voltage power supply (20) that positively charges the nozzle of the printhead either via direct wiring to the nozzle or to a ring (6) around the needle. By grounding the print bed (7) an electrical field can be attained that allows flow from the ring/nozzle to the print bed, and allows the formation of accelerating droplets of biomaterials.
Levitation Module (LM): comprises an array of ultrasonic sources (8) located underneath the print bed that are in charge of elevating the printed media (10) via sound wave interference (9).
Gantry Mechanism (GM): Given that the print bed is fixed, the printhead is moved using a gantry mechanism that provides planar and vertical motion using the horizontal axis (11) and the z axis (12).
Melt Electro writing Module (MEWM): comprises a cylinder and piston (13) which uses a linear actuator to guide melted biomaterials through a positively charged nozzle. A tubular heater (14) is employed to change the phase of the material and make it more fluidic.
Incubator control system (ICS): The system comprises 4 closed-loop controllers designed to provide on-time control of oxygen levels, as well as the levels of carbon dioxide, relative humidity and the temperature of the printing environment (15). In an embodiment, each controller is in charge of automatically measuring the real values of these parameters (17) and setting each of them to a given desired value (16). In an embodiment, a UV light source (22) is used to disinfect the printed structures of any potential pollutants.
Automatic electrospinning and electrospraying module (AEEM): This module automatically controls phase changing that occurs between electrospinning and electrospraying. This module comprises two low-level controllers (18) and a high-level controller. The shift from electrospinning to electrospraying and vice versa is done automatically via a high-level controller (21). In an embodiment, this can be achieved by setting the electrical field and the mixer to a desired voltage and to the desired fluid properties. In an embodiment, the low level controllers are in charge of controlling the fluid properties fluidic (19) using feedback from the mixer (4) and setting the high voltage to a desired value (20). The fluidic property controller works as follows:
   The viscosity and the flow rate of the final blended fluid (5) depends on the velocity profile of the actuators a-d (Vₐ, V_{b}, V_{c}, V_{d}).
   Then the controller drives the linear actuators based on the fluid profile feedback from the sensors (4).
   The control loop (19) guarantees the final blend (5) would eventually reach the desired fluidic profile.

### Applications:

In an embodiment, the fabricated tissue of the present invention can be used in the realm of drug discovery. These human tissue models can:
1. Act as a robust alternative to animal testing, addressing the ethical concerns and the often non-transferable results from animals to humans.
2. Facilitate a more accurate prediction of human physiological responses to a wide array of drug compounds.
3. Reduce the time, costs, and potential risks associated with traditional drug testing methods.
4. Furthermore, the innovation holds potential for developing personalized medicine, tissue transplantation, disease modeling, and therapeutic applications. The following list lays out some of the applications (followed by a short explanation of those applications) that can be attained by the systems/methods of the present invention:
   a. Regenerative Medicine and Tissue Engineering:
      i. Customized Organ and Tissue Printing: This technology can be used to create patient-specific organs and tissues for transplantation, reducing the risk of organ rejection.
      ii. Scaffold-Free Tissue Engineering: This technology enables the creation of three-dimensional tissue constructs without the need for traditional scaffolds, which can improve tissue integration and functionality.
      iii. Drug Testing and Development: These advanced bioprinted tissues can serve as realistic models for drug testing, reducing the reliance on animal testing and speeding up drug development.
   Biological Research:
      iv. Disease Modeling: Bioprinting can be used to create disease models for studying the progression of various illnesses and testing potential treatments.
      v. Cell Behavior Studies: Researchers can use this technology to study cell behavior, interactions, and responses to different microenvironments.
   b. Pharmaceuticals and Drug Delivery
      i. Drug Screening: Advanced bioprinted models can be used to screen and identify potential drug candidates more efficiently.
      ii. Personalized Medicine: Bioprinted tissues can be used to develop personalized drug delivery systems tailored to an individual's unique needs.
   c. Cosmetic and Dermatological Applications:
      i. Skin Tissue Printing: This technology can be used to create skin grafts for burn victims or individuals with skin disorders.
      ii. Cosmetic Testing: Cosmetic companies can use bioprinted skin models for testing one or more products' safety and efficacy.

The present invention signifies a pivotal shift in the domain of tissue engineering, merging cutting-edge technologies to provide an authentic and reliable human tissue model. With its ability to revolutionize drug testing and numerous medical applications, this invention paves the way for a more humane and precise future in regenerative medicine.

In an embodiment, the present invention relates to a method for fabricating complex human tissues, comprising:
a. providing a bio-electrospraying apparatus equipped with a pair of charged electrodes;
b. providing a cell electrospinning apparatus equipped with a nozzle tip, a high voltage supply, a pump to control flow rate, and a grounded collector;
c. providing a 3D microfluidic gradient bioprinting apparatus to be merged with the bio-electrospraying apparatus and the cell-electrospinning apparatus;
d. providing one or more of magnetic and/or acoustic levitation apparatuses;
e. embedding live cells in a biocompatible polymer to create polymer-embedded cells;
f. subjecting the polymer-embedded cells to bio-electrospraying and cell electrospinning processes to generate nano-fiber mats or micro-droplets with embedded cells;
g. layering the nano-fiber mats or micro-droplets using the 3D microfluidic gradient bioprinting apparatus to construct a three-dimensional scaffold;
h. applying magnetic and acoustic levitation to align and organize the live cells within the three-dimensional scaffold;
i. culturing the three-dimensional scaffold under suitable conditions to promote cell growth and tissue development, thereby fabricating complex human tissues.

In a variation of the method, the method further comprises a step of j. utilizing the fabricated complex human tissues for drug discovery processes to evaluate the effects of drug compounds on human physiology.

In a variation, the bio-electrospraying and cell electrospinning processes exploit an electric field between the pair of charged electrodes to draw a liquid jet, generating either droplets or continuous fibers. In a variation, the 3D microfluidic gradient bioprinting apparatus is utilized to deposit multiple types of human cell suspensions as bio-ink/biomaterials to create multi-cellular structures with an adaptable microenvironment.

In an embodiment, the one or more magnetic and acoustic levitation apparatuses are utilized to create a controlled microenvironment for spatial organization and alignment of the live cells within the three-dimensional scaffold. This spatial organization and alignment allows for tissue vascularization. In a variation, an electric field stimulation is employed to promote organized tissue engineering strategies within the three-dimensional scaffold.

In an embodiment, the present invention relates to a system for fabricating complex human tissues, the system comprising:
a. a bio-electrospraying apparatus;
b. a cell electrospinning apparatus;
c. a 3D microfluidic gradient bioprinting apparatus;
d. magnetic and acoustic levitation apparatuses;
e. a culture medium supply for promoting cell growth and tissue development; each of the bio-electrospraying apparatus, the cell electrospinning apparatus, the 3D microfluidic gradient bioprinting apparatus, and the magnetic and acoustic levitation apparatuses being synergistically connected to each other.

In a variation, the system further comprises a testing platform for evaluating the effects of drug compounds on the complex human tissues that are fabricated. In a variation, the testing platform comprises assays for evaluating drug toxicity, efficacy, and/or pharmacokinetics. In a variation, the testing platform can be used to screen compounds for further study. In a variation, the bio-electrospraying apparatus, the cell electrospinning apparatus, the 3D microfluidic bioprinting apparatus, and the magnetic and acoustic levitation apparatuses are integrated into a single automated platform for streamlined fabrication of complex human tissues.

In an embodiment, the system further comprises a control unit configured to control operational parameters of the bio-electrospraying apparatus, the cell electrospinning apparatus, the 3D microfluidic bioprinting apparatus, and the magnetic and acoustic levitation apparatuses to optimize tissue fabrication. In a variation, the control unit is a computer.

In a variation, the computer comprises artificial intelligence (AI). The AI can be used to not only suggest improvements in parameters that can be utilized for tissue fabrication, but may also suggest compounds that can be modified from the tested compounds that may give better optimized drug testing results. In a variation, the control unit (such as a computer that may comprise AI) may be further configured to monitor and adjust the culture medium supply to promote optimal cell growth and tissue development.

In an embodiment, the present invention relates to a method of ascertaining an effect of a drug on a complex human tissue, said method comprising utilizing
a. a bio-electrospraying apparatus;
b. a cell electrospinning apparatus;
c. a 3D microfluidic gradient bioprinting apparatus;
d. magnetic and acoustic levitation apparatuses; and
e. a culture medium supply for promoting cell growth and tissue development;
   to fabricate the complex human tissue, and subsequently testing the drug on the complex human tissue that has been fabricated thereby allowing one to ascertain the effect of the drug.

In a variation, the testing is performed utilizing a platform that tests for drug toxicity, efficacy, and/or pharmacokinetics. In a variation, the testing is performed utilizing a platform that tests for drug toxicity, efficacy, and pharmacokinetics. In a variation, the complex human tissue comprises vascularized tissue. In a variation, the platform utilizes a computer to compare test results relative to other tested drug compounds. In a variation, the computer uses AI to suggest new compounds to be tested.

The following references are incorporated by reference in their entireties.
1. Chapter Twelve—Combinatorial approaches in electrospinning, electrospraying, and 3D printing for biomedical applications, Ameer, Jimna Mohamed et al., Biomedical Applications of Electrospinning and Electrospraying, 2021, pp. 353-373.
2. Bio-electrospraying assessment toward in situ chondrocyte-laden electrospun scaffold fabrication, Semitela, Angela et al., J. Tissue Eng. 2022, 13, pp. 1-17.
3. Combination of 3D Printing and Electrospinning Techniques for Biofabrication, Yang, Dan-Lei et al., Advanced Materials Technology, 2022, 7(7), 2270031.
4. Electrospinning and Electrospraying with Cells for Applications in Biomanufacturing, Zhao, Qilong, 2021, 11, 2141003.
5. Maurmann, N., Sperling, LE., Pranke, P. (2018). Electrospun and Electrosprayed Scaffolds for Tissue Engineering. In: Chun, H., Park, C., Kwon, I., Khang, G. (eds) Cutting-Edge Enabling Technologies for Regenerative Medicine. Advances in Experimental Medicine and Biology, vol 1078. Springer, Singapore. https://doi.org/10.1007/978-981-13-0950-2_6. Magnetic Levitational Assembly for Living Material Fabrication, Tasoglu, Savas et al., Adv Healthc Mater., 2015, 4(10), 1469-1476.
7. Fabrication of Tunable 3D Cellular Structures in High Volume Using Magnetic Levitation Guided Assembly, Onbas, Rabia et al., ACS Appl Bio Mater. 2021 Feb. 15; 4(2):1794-1802.
8. A sound approach to advancing healthcare systems: the future of biomedical acoustics, Rufo, Joseph et al., Nature Communications volume 13, Article number: 3459 (2022).
9. Development and Analysis of a Novel Magnetic Levitation System with a Feedback Controller for Additive Manufacturing Applications, Kumar, Parichit et al., Actuators 2022, 11(12), 364.
10. Jayarajan, V., Auguste, J. O., Gene, K. A., Auguste, L., Nunez, C., Marcinowski, B. and Jayasinghe, S. N., 2023. Bio-electrospraying 3-D Organotypic Human Skin Cultures. Small, p. 2304940.

We claim:

1. A method for fabricating complex human tissues, comprising:
a. providing a bio-electrospraying apparatus equipped with a pair of charged electrodes;
b. providing a cell electrospinning apparatus equipped with a nozzle tip, a high voltage supply, a pump to control flow rate, and a grounded collector;
c. providing a 3D microfluidic gradient bioprinting apparatus to be merged with the bio-electrospraying apparatus and the cell-electrospinning apparatus;
d. providing one or more of magnetic and/or acoustic levitation apparatuses;
e. embedding live cells in a biocompatible polymer to create polymer-embedded cells;
f. subjecting the polymer-embedded cells to bio-electrospraying and cell electrospinning processes to generate nano-fiber mats or micro-droplets with embedded cells;
g. layering the nano-fiber mats or micro-droplets using the 3D microfluidic gradient bioprinting apparatus to construct a three-dimensional scaffold;
h. applying magnetic and acoustic levitation to align and organize the live cells within the three-dimensional scaffold;
i. culturing the three-dimensional scaffold under suitable conditions to promote cell growth and tissue development, thereby fabricating complex human tissues.

2. The method of claim 1, further comprising a step of
j. utilizing the fabricated complex human tissues for drug discovery processes to evaluate the effects of drug compounds on human physiology.

3. The method of claim 1, wherein the bio-electrospraying and cell electrospinning processes exploit an electric field between the pair of charged electrodes to draw a liquid jet, generating either droplets or continuous fibers.

4. The method of claim 1, wherein the 3D microfluidic gradient bioprinting apparatus is utilized to deposit multiple types of human cell suspensions as bio-ink/biomaterials to create multi-cellular structures with an adaptable microenvironment.

5. The method of claim 1, wherein the one or more magnetic and acoustic levitation apparatuses are utilized to create a controlled microenvironment for spatial organization and alignment of the live cells within the three-dimensional scaffold.

6. The method of claim 1, wherein electric field stimulation is employed to promote organized tissue engineering strategies within the three-dimensional scaffold.

7. A system for fabricating complex human tissues, the system comprising:
a. a bio-electrospraying apparatus;
b. a cell electrospinning apparatus;
c. a 3D microfluidic gradient bioprinting apparatus;
d. magnetic and acoustic levitation apparatuses;
e. a culture medium supply for promoting cell growth and tissue development; each of the bio-electrospraying apparatus, the cell electrospinning apparatus, the 3D microfluidic gradient bioprinting apparatus, and the magnetic and acoustic levitation apparatuses being synergistically connected to each other.

8. The system of claim 7, wherein the system further comprises f. a testing platform for evaluating the effects of drug compounds on the complex human tissues that are fabricated.

9. The system of claim 8, wherein the testing platform comprises assays for evaluating drug toxicity, efficacy, and/or pharmacokinetics.

10. The system of claim 7, wherein the bio-electrospraying apparatus, the cell electrospinning apparatus, the 3D microfluidic bioprinting apparatus, and the magnetic and acoustic levitation apparatuses are integrated into a single automated platform for streamlined fabrication of complex human tissues.

11. The system of claim 7, further comprising a control unit configured to control operational parameters of the bio-electrospraying apparatus, the cell electrospinning apparatus, the 3D microfluidic bioprinting apparatus, and the magnetic and acoustic levitation apparatuses to optimize tissue fabrication.

12. The system of claim 11, wherein the control unit is a computer.

13. The system of claim 12, wherein the computer comprises artificial intelligence (AI).

14. The system of claim 11, wherein the control unit is further configured to monitor and adjust the culture medium supply to promote optimal cell growth and tissue development.

15. A method of ascertaining an effect of a drug on a complex human tissue, said method comprising utilizing
a. a bio-electrospraying apparatus;
b. a cell electrospinning apparatus;
c. a 3D microfluidic gradient bioprinting apparatus;
d. magnetic and acoustic levitation apparatuses; and
e. a culture medium supply for promoting cell growth and tissue development;
to fabricate the complex human tissue, and subsequently testing the drug on the complex human tissue that has been fabricated thereby allowing one to ascertain the effect of the drug.

16. The method of claim 15, wherein the testing is performed utilizing a platform that tests for drug toxicity, efficacy, and/or pharmacokinetics.

17. The method of claim 16, wherein the testing is performed utilizing a platform that tests for drug toxicity, efficacy, and pharmacokinetics.

18. The method of claim 17, wherein the complex human tissue comprises vascularized tissue.

19. The method of claim 16, wherein the platform utilizes a computer to compare test results relative to other tested drug compounds.

20. The method of claim 19, wherein the computer uses AI to suggest new compounds to be tested.
